(19) United States
(12) Patent Application Publication
(10) Pub. No.: US 2025/0353823 A1
(43) Pub. Date: Nov. 20, 2025

(54) PROCESS FOR THE PREPARATION OF ALKOXYLATED 2,5-DIHYDROFURAN

(71) Applicant: DSM IP ASSETS B.V., Maastricht (NL)

(72) Inventors: Werner BONRATH, Kaiseraugst (CH); Earl Lawrence Vincent GOETHEER, Den Haag (NL); Roman GOY, Kaiseraugst (CH); Marcel JORAY, Kaiseraugst (CH); Roman LATSUZBAIA, Den Haag (NL)

(21) Appl. No.: 18/873,525

(22) PCT Filed: Jun. 9, 2023
(86) PCT No.: PCT/EP2023/065512

§ 371 Date: Dec. 10, 2024

(30) Foreign Application Priority Data
Jun. 15, 2022 (EP) 22179109.8

(51) Int. Cl. C07D 307/32; C25B 3/05; C25B 3/07

(52) U.S. Cl. CPC C07D 307/32; C25B 3/05; C25B 3/07

(57) ABSTRACT

The present invention relates to a process for the preparation of alkoxylated 2.5-dihydrofuran. The process is carried out in an electrochemical cell reactor with a vertical flow.

The present invention relates to a novel process for the preparation of alkoxylated 2,5-dihydrofuran. The process is carried out electrochemically.

Alkoxylated 2,5-dihydrofuran, which are the compounds of formula (I)

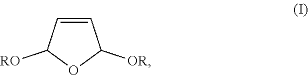

wherein R is a linear or branched C₁-C₆ alkyl group, are very useful compounds.

They can be used as such, or they can be used as intermediates in organic synthesis (for example as intermediates in the production of carotenoids, such as intermediates for the carotenoid production).

Due to the importance of these compounds, there is always a need for an improved synthesis for these compounds.

WO2006/100289 discloses a process to produce 2,5-dihydrofuran derivatives by electrochemical oxidation in the presence of a C₁- to C₆-monoalkyl alcohol. An anode and cathode made from graphite is used and a yield of 46 percent, of 2,5-dimethoxy-2,5-dihydro-furan was obtained. The selectivity was 51 percent.

The alkoxylated 2,5-dihydrofuran are produced using the compounds of formula (II), which are preferred in its Z form (as shown below) as starting material as shown in the following scheme:

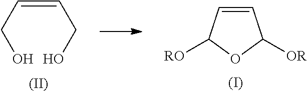

Surprisingly, it was found when the process was carried out under specific conditions, the yield of the alkoxylated 2,5-dihydrofuran (compound of formula (I)) was increased significantly.

Surprisingly, it was found when a specific arrangement of an electrochemical reactor (cell). is used, an increased yield (significantly higher than in the prior art) is obtained.

It was found out when an electrochemical reactor with a vertical flow is used, an increased result can be obtained. By the term “vertical flow” it is meant the flow is from the bottom to the top of the electrochemical reactor or from the top to the bottom of the electrochemical reactor (preferably from the bottom to the top of the electrochemical reactor).

Therefore, the present invention relates to a process (P) for the preparation of a compound of formula (I)

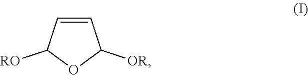

wherein R is a linear or branched C₁-C₆ alkyl group,
which comprises electrochemically reacting the compound of formula (II) in Z-form

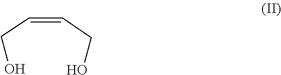

with at least one mono alcohol of formula (III)

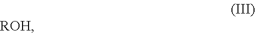

wherein R has the same meaning as in compound of formula (I)
characterized in that
the process is carried out in an electrochemical reactor with a vertical flow.

As it can be seen from formula (II) is in Z-form, when used in the process according to the present invention.

But it is possible that a small amount of the compound of formula (II) in its E-form can be used as well. The E-form can be present in amount of less than 5wt-%, based on the total weight of the compound of formula (II) in the process.

Preferred compounds of formula (I) are those wherein
R is —CH₃ or —CH₂CH₃.

More preferred is the compound of formula (I) wherein
R is —CH₃.

Therefore, the present invention relates to a process (P1), which is process (P), wherein compounds of formula (I) wherein
R is —CH₃ or —CH₂CH₃,
are used.

Therefore, the present invention relates to a process (P1′), which is process (P), wherein the compound of formula (I) wherein
R is —CH₃
is used.

The process of the present invention is usually carried out in non-aqueous medium.

In the context of the present invention the term “non-aqueous” means that less than 50wt-%, based on the total weight of the non-aqueous media, of water can be present in the non-aqueous media.

Usually, the term “non-aqueous” means that less than 20wt-%, based on the total weight of the non-aqueous media, of water can be present in the non-aqueous media.

Therefore, the present invention relates to a process (P2), which is process (P), (P1) or (P1′), wherein the process is carried out in a non-aqueous medium.

The non-aqueous medium is (or comprises) usually and preferably at least one linear or branched C₁-C₁₀ alcohol (preferably at least one linear or branched C₁-C₆ alcohol, more preferably ethanol or methanol, most preferably methanol).

This means that the mono alcohol of formula (III) can also serve as non-aqueous medium, or it can be a mixture of other alcohol and the mono alcohol of formula (III). It is preferred that the mono alcohol of formula (III) is also used as the non-aqueous medium.

Therefore, the present invention relates to a process (P2′), which is process (P2), wherein the non-aqueous medium is at least one linear or branched C₁-C₁₀ alcohol.

Therefore, the present invention relates to a process (P2″), which is process (P2), wherein the non-aqueous medium is the mono alcohol of formula (III)

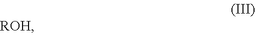

wherein R is a C₁-C₆ alkyl group.

Therefore, the present invention relates to a process (P2″), which is process (P2), wherein the non-aqueous medium is at least one alcohol is chosen from the group consisting of methanol, ethanol, n-propanol and isopropanol.

Therefore, the present invention relates to a process (P2″′), which is process (P2), wherein the non-aqueous medium is methanol and/or ethanol.

Therefore, the present invention relates to a process (P2″″), which is process (P2), wherein the non-aqueous medium is methanol.

The at least one alcohol of formula (III) is used in an amount of at least 2 mol-equivalents regarding the compound of formula (II). This means that this alcohol is always present in that amount at least, when not used as non-aqueous medium.

Of course, it is also possible that the non-aqueous media is the at least one alcohol of the compound of formula (III).

Therefore, the present invention relates to a process (P3), which is process (P), (P1), (P1′), (P2), (P2′), (P2″), (P2″′) or (P2″″), wherein the at least one alcohol of formula (III) is used in an amount of at least 2 mol-equivalents regarding the compound of formula (II).

An essential feature of the present invention is that the process according to the present invention is carried out in an electrochemical reactor with a vertical flow a better result can be obtained. This means the flow of the reaction mixture can be from bottom to top or from top to bottom of the electrochemical reactor (preferably from bottom to top of the electrochemical reactor). This is usually done by a pumping system.

The size and the form/shape (and therefore also the volume) of the electrochemical reactor can vary. The size and the form/shape (as well as the volume) of the electrochemical reactor is not an essential feature.

A very common and also a preferred form is a 3D rectangular shape (cuboid).

Therefore, the present invention relates to a process (P4), which is process (P), (P1), (P1′), (P2), (P2′), (P2″), (P2″′), (P2″″) or (P3), wherein the process is carried out in a 3D rectangular shaped electrochemical reactor.

The flow rate of the starting material can vary. This depends on the size, form and volume of the cuboid electrochemical reactor.

An usual (vertical) flow rate is at least 10 mL/min. An usual and preferred range is 10 mL/min to 1000 mL/min.

Therefore, the present invention relates to a process (P5), which is process (P), (P1), (P1′), (P2), (P2′), (P2″), (P2″′), (P2″″), (P3) or (P4), wherein (vertical) flow rate is at least 10 mL/min.

Therefore, the present invention relates to a process (P5′), which is process (P), (P1), (P1′), (P2), (P2′), (P2″), (P2″′), (P2″″), (P3) or (P4), wherein (vertical) flow rate is 10 mL/min to 1000 mL/min.

The electrodes (cathode and electrode) used in the process according to the present invention can be made from any commonly used material only or it can be made from more than one material (like a metal on a carrier material or a metal oxide on a carrier material).

Within the process, the target product is formed on the anode and dihydrogen is evolved at the cathode. Therefore, it is advantageously to use a metal or alloy instead of graphite as cathode (graphite is commonly used in prior art) since they are more active in dihydrogen production. An additional advantage of using a metal or a metal alloy as cathode is a significantly reduced cell potential, which results in energy savings for the process.

Examples of cathode materials which may be used are metals (such as iron, or noble metals, e.g. platinum), graphite or metal alloys (e.g. steel).

As stated above it in a preferred embodiment of the present invention the cathode is not made from graphite.

Materials which are stable under the conditions of the electrolysis are employed for the anode, examples of such materials being noble metals (e.g. platinum), oxides (e.g. ruthenium dioxide on titanium), graphite, highly oriented pyrolytic graphite (HOPG), boron-doped diamond (BDD), dimensionally stable anodes (DSA) and glassy-carbon.

Therefore, the present invention relates to a process (P6), which is process (P), (P1), (P1′), (P2), (P2′), (P2″), (P2″′), (P2″″), (P3), (P4), (P5) or (P5′), wherein the cathode is made from materials chosen from the group consisting of metals (such as iron, or noble metals, e.g. platinum), graphite and metal alloys (e.g. steel).

Therefore, the present invention relates to a process (P6′), which is process (P), (P1), (P1′), (P2), (P2′), (P2″), (P2″′), (P2″″), (P3), (P4), (P5) or (P5′), wherein the cathode is not made from graphite.

Therefore, the present invention relates to a process (P6″), which is process (P), (P1), (P1′), (P2), (P2′), (P2″), (P2″′), (P2″″), (P3), (P4), (P5) or (P5′), wherein the cathode is made from materials chosen from the group consisting of metals (such as iron, or noble metals, e.g. platinum) and metal alloys (e.g. steel).

Therefore, the present invention relates to a process (P7), which is process (P), (P1), (P1′), (P2), (P2′), (P2″), (P2″′), (P2″″), (P3), (P4), (P5), (P5′), (P6), (P6′) or (P6″), wherein the anode is made from materials chosen from the group consisting of noble metals, oxides, graphite, highly oriented pyrolytic graphite (HOPG), boron-doped diamond (BDD), dimensionally stable anodes (DSA) and glassy-carbon.

The electrodes can be in any usual form. Such forms can be a plate, wire, a rod, a cell, a mesh, a grid, a sponge, or any other design, which is usually used.

Therefore, the present invention relates to a process (P8), which is process (P), (P1), (P1′), (P2), (P2′), (P2″), (P2″′), (P2″″), (P3), (P4), (P5), (P5′), (P6), (P6′), (P6″) or (P7), wherein the electrodes are in the form of a plate, wire, a rod, a cell, a mesh, a grid, a sponge, or any other design, which is usually used.

The size of the electrode used in the process according to the present invention can vary and it depends on the size, the form and the structure of the electrochemical reactor (cell). A usual size is a least 10 cm² (per cell). The upper limit of the electrode is not so critical. Usually (but not necessarily), the cathode and the anode do have the same size.

Therefore, the present invention relates to a process (P9), which is process (P), (P1), (P1′), (P2), (P2′), (P2″), (P2″′), (P2″″), (P3), (P4), (P5), (P5′), (P6), (P6′), (P6″), (P7) or (P8), wherein the electrodes have a size of at least 10 cm².

Therefore, the present invention relates to a process (P10), which is process (P), (P1), (P1′), (P2), (P2′), (P2″), (P2″′), (P2″″), (P3), (P4), (P5), (P5′), (P6), (P6′), (P6″), (P7), (P8) or (P9), wherein the cathode and the anode have the same size.

The reaction medium usually and preferably comprises at least one electrolyte. That can be added to the reaction medium in the form of a salt and/or in form of an acid. Any commonly known and commonly used electrolyte can be used with the exception of phosphoric acid and/or any salt, thereof.

Suitable supporting electrolytes are i.e. HCl, H₂SO₄, Na₂SO₄, NaCl, sodium dodecyl sulfate, methyltributylammonium methylsulfate, triethylammonium bisulfate, tetrabutylammonium bisulfate, tetramethylammonium bisulfate, tetrabutylammonium acetate (NBu₄OAc), tetrabutylammonium sulfate, tetrabutylammonium hexafluorophosphate, tetrabutylammonium tetrafluoroborate, methanesulfonic acid, ammonium bisulfate, tetrabutylphosphonium methanesulfonate, 1-methylimidazolium bisulfate, tetrabutylammonium perchlorate and LiClO₄.

Usually, a concentration of up to 2 M of the at least one electrolyte is used (preferably 0.01-1 M, more preferably 0.1 to 0.5 M, 02.-0.5M).

In preferred embodiment, the electrolyte is not phosphoric acid and/or a salt, thereof.

Therefore, the present invention relates to a process (P11), which is process which is process (P), (P1), (P1′), (P2), (P2′), (P2″), (P2″′), (P2″″), (P3), (P4), (P5), (P5′), (P6), (P6′), (P6″), (P7), (P8), (P9) or (P10), wherein the process is carried out in the presence of at least one electrolyte.

Therefore, the present invention relates to a process (P11′), which is process (P11), wherein the at least one electrolyte is not phosphoric acid and/or a salt, thereof.

Therefore, the present invention relates to a process (P11″), which is process (P11) or (P11′), wherein the at least one electrolyte is chosen from the group consisting of HCl, H₂SO₄, Na₂SO₄, NaCl, sodium dodecyl sulfate, methyltributylammonium methylsulfate, triethylammonium bisulfate, tetrabutylammonium bisulfate, tetramethylammonium bisulfate, tetrabutylammonium acetate (NBu₄OAc), tetrabutylammonium sulfate, tetrabutylammonium hexafluorophosphate, tetrabutylammonium tetrafluoroborate, methanesulfonic acid, ammonium bisulfate, tetrabutylphosphonium methanesulfonate, 1-methylimidazolium bisulfate, tetrabutylammonium perchlorate and LiCIO4.

Therefore, the present invention relates to a process (P11″′), which is process (P11), (P11′) of (P11″), wherein the at least one electrolyte is used a concentration of up to 2 M of the at least one electrolyte (preferably 0.01-1 M, more preferably 0.1 to 0.5 M).

The pH value of the reaction medium of the process according to the present invention at the start of the process is preferably between 0 and 7.

Therefore, the present invention relates to a process (P12), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″) or (P11″′), wherein the reaction medium has a pH value of 0 to 7 at the start of the process.

The process according to the present invention is carried out at a temperature range of 0° C. to 75° C. (preferably 10° C. to 60° C., more preferably, 15° C. to 40° C.).

Therefore, the present invention relates to a process (P13), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′) or (P12), wherein the reaction is carried out at a temperature range of 0 to 75° C.

Therefore, the present invention relates to a process (P13′), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′) or (P12), wherein the reaction is carried out at a temperature range of 10° C. to 60° C.

Therefore, the present invention relates to a process (P13″), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′) or (P12), wherein the reaction is carried out at a temperature range of 15° C. to 40° C.

The process according to the present invention is usually carried out at ambient pressure.

Therefore, the present invention relates to a process (P14), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′) or (P13″), wherein the reaction is carried out at ambient pressure.

Depending on the cell, the process according to the present invention can be carried out batchwise or in a continuous way. The continuous process is preferred.

Therefore, the present invention relates to a process (P15), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″) or (P14), wherein the reaction is carried out batchwise or in a continuous way.

Therefore, the present invention relates to a process (P15′), which is process (P), (P1), (P1′), (P2), (P2′), (P2″), (P2″′), (P2″′), (P3), (P4), (P5), (P5′), (P6), (P6′), (P6″), (P7), (P8), (P9), (P10), (P11), (P11′), (P11″), (P11″′), (P12), (P13), (P13′), (P13″) or (P14), wherein the reaction is carried out a continuous way.

The current density used in the process according to the present invention is preferably between 1-1000 mA/cm². Preferably, 10-1000 mA/cm², more preferably 20-1000 mA/cm²).

Therefore, the present invention relates to a process (P16), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15) or (P15′), wherein the reaction is carried out at a current density of between 1-1000 mA/cm².

Therefore, the present invention relates to a process (P16′), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15) or (P15′), wherein the reaction is carried out at a current density of between 10-1000 mA/cm².

Therefore, the present invention relates to a process (P16″), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15) or (P15′), wherein the reaction is carried out at a current density of between 20-1000 mA/cm².

The electrical potential between the anode and cathode may be 12 V or less. A suitable range is 0.5-12 V, preferred is 0.5-10 V; more preferred is 0.5-8 V; most preferred is 1-8 V.

Therefore, the present invention relates to a process (P17), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15), (P15′), (P16), (P16′) or (P16″), wherein the electrical potential between the anode and cathode is 12 V or less.

Therefore, the present invention relates to a process (P17′), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15), (P15′), (P16), (P16′) or (P16″), wherein the electrical potential between the anode and cathode is 0.5-12 V.

Therefore, the present invention relates to a process (P17″), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15), (P15′), (P16), (P16′) or (P16″), wherein the electrical potential between the anode and cathode is 0.5-10 V.

Therefore, the present invention relates to a process (P17″), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15), (P15′), (P16), (P16′) or (P16″), wherein the electrical potential between the anode and cathode is 0.5-8 V.

Therefore, the present invention relates to a process (P17″), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15), (P15′), (P16), (P16′) or (P16″), wherein the electrical potential between the anode and cathode is 1-8 V.

The process according to the present invention can be carried out in galvanostatic or potentiostatic mode.

Therefore, the present invention relates to a process (P18), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15), (P15′), (P16), (P16′), (P16″), (P17), (P17′), (P17″), (P17″′) or (P17″), wherein the process is carried out in galvanostatic mode.

Therefore, the present invention relates to a process (P19), which is process (P), (P1), (P1′), (P2), (P2′), (P2″), (P2″′), (P2″″), (P3), (P4), (P5), (P5′), (P6), (P6′), (P6″), (P7), (P8), (P9), (P10), (P11), (P11′), (P11″), (P11″′), (P12), (P13), (P13′), (P13″), (P14), (P15), (P15′), (P16), (P16′), (P16″), (P17), (P17′), (P17″), (P17″′), (P17″) or (P18), wherein the process is carried out in potentiostatic mode.

The reaction products (the compound of formula (I)) can be isolated from the reaction medium using commonly methods.

The following examples serve to illustrate the invention. If not otherwise stated all parts are given are related to the weight and the temperature is given in ° C.

### EXAMPLES

### Example 1

Electrochemical oxidation reaction of 1 M Z-2-butene-1,4-diol (compound of formula (la) to 2,5-dihydro-2,5-dimethoxyfuran was carried out in an undivided flow-cell (V=10 mL, surface area 100 cm², 1 mm electrode distance) in methanol, 5.6 wt % methyltributylammonium methylsulfate was used as electrolyte. Graphite (100 cm2) electrode was used as anode and stainless steel (100 cm²) was used as cathode. Electrolysis was carried out galvanostatically by applying current of 100 mAcm⁻² at 20° C. (measured cell potential 5.9 V). The reaction mixture was pumped vertically from bottom to top with a flowrate of 400 mL/min through the flow-cell. After 90 min, a conversion of 96% Z-2-butene-1,4-diol was achieved, and the methanol was distilled of. The evaporated reaction mixture was distilled to get DMDF in the distillate with an overall 70% yield and a 62% Faraday efficiency was achieved. In the residue quantitative amount of MTBS was obtained with a purity of 73%.

### Example 2

Electrochemical oxidation reaction of 2 M Z-2-butene-1,4-diol (compound of formula (la) to 2,5-dihydro-2,5-dimethoxyfuran was carried out in an undivided flow-cell (V=10 mL, surface area 100 cm², 1 mm electrode distance) in methanol, 5.1 wt % methyltributylammonium methylsulfate was used as electrolyte. Graphite (100 cm²) electrode was used as anode and stainless steel (100 cm²) was used as cathode. Electrolysis was carried out galvanostatically by applying current of 100 mAcm⁻² at 20° C. (measured cell potential 6.5 V). The reaction mixture was pumped vertically from bottom to top with a flowrate of 400 mL/min through the flow-cell. After 190 min, a conversion of 97% Z-2-butene-1,4-diol was achieved. The reaction mixture had a content of 63% DMDF and a 52% Faraday efficiency was achieved.

### Example 3

Electrochemical oxidation reaction of 1 M Z-2-butene-1,4-diol (compound of formula (la) to 2,5-dihydro-2,5-dimethoxyfuran was carried out in an undivided flow-cell (V=10 mL, surface area 100 cm², 1 mm electrode distance) in methanol, 8.2 wt % triethylammonium bisulfate was used as electrolyte. Graphite (100 cm²) electrode was used as anode and graphite (100 cm²) was used as cathode. Electrolysis was carried out galvanostatically by applying current of 100 mAcm⁻² at 20° C. (measured cell potential 6.2 V). The reaction mixture was pumped vertically from bottom to top with a flowrate of 400 mL/min through the flow-cell. After 90 min, a conversion of 97% Z-2-butene-1,4-diol was achieved. The reaction mixture had a content of 66% DMDF and a 58% Faraday efficiency was achieved.

### Example 4

Electrochemical oxidation reaction of 1 M Z-2-butene-1,4-diol (compound of formula (la) to 2,5-dihydro-2,5-dimethoxyfuran was carried out in an undivided flow-cell (V=10 mL, surface area 100 cm², 1 mm electrode distance) in methanol, 8.2 wt % triethylammonium bisulfate was used as electrolyte. Graphite (100 cm²) electrode was used as anode and graphite (100 cm²) was used as cathode. Electrolysis was carried out galvanostatically by applying current of 150 mAcm⁻² at 20° C. (measured cell potential 7.3 V). The reaction mixture was pumped vertically from bottom to top with a flowrate of 400 mL/min through the flow-cell. After 60 min, a conversion of 98% Z-2-butene-1,4-diol was achieved. The reaction mixture had a content of 68% DMDF and a 60% Faraday efficiency was achieved.

### Example 5

Electrochemical oxidation reaction of 1 M Z-2-butene-1,4-diol (compound of formula (la) to 2,5-dihydro-2,5-dimethoxyfuran was carried out in an undivided flow-cell (V=10 mL, surface area 100 cm², 1 mm electrode distance) in methanol, 8.2 wt % triethylammonium bisulfate was used as electrolyte. Graphite (100 cm²) electrode was used as anode and stainless steel (100 cm²) was used as cathode. Electrolysis was carried out galvanostatically by applying current of 150 mAcm⁻² at 20° C. (measured cell potential 6.0 V). The reaction mixture was pumped vertically from bottom to top with a flowrate of 400 mL/min through the flow-cell. After 60 min, a conversion of 98% Z-2-butene-1,4-diol was achieved. The reaction mixture had a content of 65% DMDF and a 57% Faraday efficiency was achieved.

### Example 6

Electrochemical oxidation reaction of 0.5 M BED (Z-2-butene-1,4-diol) to DMDF (2,5-dihydro-2,5-dimethoxyfuran) was carried out in an undivided flow-cell (V=10 mL, surface area 100 cm², 1 mm electrode distance) in methanol, 3.1 wt % Sodium dodecyl sulfate was used as electrolyte. Graphite (100 cm²) electrode was used as anode and stainless steel (100 cm²) was used as cathode. Electrolysis was carried out galvanostatically by applying current of 50 mAcm⁻² at 20° C. (measured cell potential 5.6 V). The reaction mixture was pumped vertically from bottom to top with a flowrate of 400 mL/min through the flow-cell. After 360 min, a conversion of 98% BED was achieved with an overall 70% yield DMDF and a 61% Faraday efficiency was achieved.

### Examples 7-15

Electrochemical oxidation reaction of 0.5 M BED (Z-2-butene-1,4-diol) to DMDF (2,5-dihydro-2,5-dimethoxyfuran) was carried out in an undivided flow-cell (V=1 mL, surface area 10 cm², 1 mm electrode distance) in methanol, using various electrolytes. Graphite (10 cm²) electrode was used as anode and various electrode materials (10 cm²) were used as cathode. Electrolysis was carried out galvanostatically by applying current between 17-50 mAcm⁻² at 20° C. (varying cell potentials). The reaction mixture was pumped vertically from bottom to top with a flowrate of 50 mL/min through the flow-cell.

| | | | Current | Cell | Conversion | Content | Faraday |
|---|---|---|---|---|---|---|---|
| | | | density | potential | BED | DMDF | efficiency |
| Exp | Electrolyte | Cathode | [mA/cm²] | [V] | [%] | [%] | [%] |
| 7 | 3.1 wt % | Stainless steel | 34 | 4.3 | 81 | 36 | 37 |
| | 1-Methylimidazolium bisulfate | | | | | | |
| 8 | 3 wt % Sodium dodecyl sulfate | Platinum | 34 | 5.4 | 90 | 70 | 80 |
| 9 | 3 wt % Sodium dodecyl sulfate | Stainless steel | 34 | 5.4 | 95 | 72 | 77 |
| 10 | 3 wt % Tetramethylammonium | Platinum | 17 | 4.0 | 94 | 53 | 57 |
| | bisulfate | | | | | | |
| 11 | 2 wt % Sulfuric acid | Platinum | 34 | 3.8 | 56 | 43 | 42 |
| 12 | 3 wt % Methyltributylammonium | Graphite | 17 | 4.2 | 98 | 71 | 80 |
| | methylsulfate | | | | | | |
| 13 | 3 wt % Tetrabutylammonium | Graphite | 17 | 4.3 | 92 | 45 | 53 |
| | bisulfate | | | | | | |
| 14 | 3 wt % Tetrabutylammonium | Platinum | 17 | 4.7 | 97 | 56 | 60 |
| | bisulfate | | | | | | |
| 15 | 6 wt % Tetrabutylammonium | Platinum | 34 | 4.3 | 95 | 56 | 61 |
| | bisulfate | | | | | | |

1. Process for the preparation of a compound of formula (I) 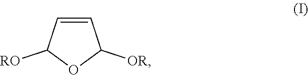
wherein R is a linear or branched C₁-C₆ alkyl group,
which comprises electrochemically reacting the compound of formula (II) in Z-form 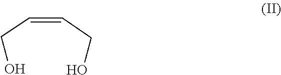
with at least one mono alcohol of formula (III) 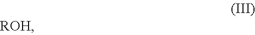
wherein R has the same meaning as in compound of formula (I)
wherein
the process is carried out in electrochemical reactor with a vertical flow.

2. Process according to claim 1, wherein
R is —CH₃ or —CH₂CH₃.

3. Process according to claim 1, wherein
R is —CH₃.

4. Process according to claim 1, wherein the process is carried out in a non-aqueous medium.

5. Process according to claim 4, wherein the at least one alcohol of formula (III) is used in an amount of at least 2 mol-equivalents in regard to the compound of formula (II).

6. Process according to claim 4, wherein the non-aqueous medium is (or comprises) at least one linear or branched C₁-C₁₀ alcohol.

7. Process according to claim 4, wherein the non-aqueous medium is the mono alcohol of formula (III) 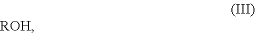
wherein R is a linear or branched C₁-C₆ alkyl group.

8. Process according to claim 1, wherein the process is carried out in a cuboid electrochemical reactor.

9. Process according to claim 1, wherein the cathode is not made from graphite.

10. Process according to claim 1, wherein the cathode is made from materials chosen from the group consisting of metals and metal alloys.

11. Process according to claim 1, wherein the anode is made from materials chosen from the group consisting of noble metals, oxides, graphite, highly oriented pyrolytic graphite (HOPG), boron-doped diamond (BDD), dimensionally stable anodes (DSA) and glassy-carbon

12. Process according to claim 1, wherein the current density applied in the process is between 1-1000 mA/cm².

13. Process according to claim 1, wherein the process is carried out in the presence of at least one supporting electrolyte.

14. Process according to claim 13, wherein at least one electrolyte is not phosphoric acid and/or a salt, thereof.

15. Process according to claim 13, wherein the at least one electrolyte is chosen from the group consisting of HCl, H₂SO₄, Na₂SO₄, NaCl, sodium dodecyl sulfate, methyltributylammonium methylsulfate, triethylammonium bisulfate, tetrabutylammonium bisulfate, tetramethylammonium bisulfate, tetrabutylammonium acetate (NBu₄OAc), tetrabutylammonium sulfate, tetrabutylammonium hexafluorophosphate, tetrabutylammonium tetrafluoroborate, methanesulfonic acid, ammonium bisulfate, tetrabutylphosphonium methanesulfonate, 1-methylimidazolium bisulfate, tetrabutylammonium perchlorate and LiCIO₄.

16. Process according to claim 1, wherein the reaction is carried out batchwise or in a continuous way.
